(19) United States
(12) Patent Application Publication
(10) Pub. No.: US 2026/0290519 A1
(43) Pub. Date: Sep. 24, 2026

(54) IDENTIFYING NUCLEATING AGENTS USING MACHINE LEARNING

(71) Applicant: Google LLC, Mountain View, CA (US)

(72) Inventors: Muratahan Aykol, San Jose, CA (US); Ekin Dogus Cubuk, Berkeley, CA (US)

(21) Appl. No.: 19/474,249

(22) PCT Filed: Apr. 12, 2024
(86) PCT No.: PCT/US2024/024352

§ 371 Date: Oct. 10, 2025

Related U.S. Application Data
(60) Provisional application No. 63/458,820, filed on Apr. 12, 2023.

(51) Int. Cl. G16C 20/30; G06N 3/045; G16C 20/20; G16C 20/64; G16C 20/70; G16C 60/00

(52) U.S. Cl. CPC G16C 20/30; G06N 3/045; G16C 20/20; G16C 20/64; G16C 20/70; G16C 60/00

(57) ABSTRACT

Methods, systems, and apparatus, including computer programs encoded on a computer storage medium, for selecting a nucleating agent for synthesis of a target material. According to one aspect, a method comprises: receiving data identifying a target material; generating an embedding of the target material in a latent space using an embedding machine learning model; and selecting one or more nucleating agents for the target material, from a set of candidate nucleating agents, based at least in part on the embedding of the target material in the latent space.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a system implemented as computer programs on one or more computers in one or more locations that can select a nucleating agent for synthesizing a target material.

A “material” is any substance or mixture of substances that composes a physical entity, e.g., a material can be a metal (e.g., an alloy), or a ceramic, or a polymer, or a composite, or a molecular crystal.

“Nucleation” is a process that marks the initial step in the formation of a new phase of a material, e.g., as a particular arrangement of atoms (or molecules) with various chemical identities distributed in space in a periodic, translational structure, also known as a “crystal structure.” In nucleation, particles of the material start to form and dissolve through a stochastic process that involves the appearance of small regions, or “nuclei,” of the new phase which are thermodynamically more stable under the current conditions than the existing phase.

A “nucleating agent” is a substance added to a material to promote nucleation. Nucleating agents work by providing sites (nucleation sites) on which the phase (e.g., crystal phase) of the material can form more easily and rapidly than in the bulk material (i.e., existing phase), thus inducing and/or accelerating the formation of the phase of the material.

An “embedding” of an entity (e.g., a material) is a representation of the entity as an ordered collection of numerical values, e.g., a vector, matrix, or other tensor of numerical values.

A first neural network can be referred to as a “subnetwork” of a second neural network if the first neural network is included in the second neural network.

According to one aspect there is provided a method performed by one or more computers, the method comprising: receiving data identifying a target material; generating an embedding of the target material in a latent space using an embedding machine learning model; and selecting one or more nucleating agents for the target material, from a set of candidate nucleating agents, based at least in part on the embedding of the target material in the latent space.

In some implementations, the data identifying the target material comprises: (i) data identifying a chemical composition of the target material, and (ii) data identifying a crystal structure of the target material.

In some implementations, generating the embedding of the target material in the latent space using the embedding machine learning model comprises processing a model input comprising the data identifying the target material using the embedding machine learning model and in accordance with values of a set of embedding machine learning model parameters to generate the embedding of the target material.

In some implementations, the embedding machine learning model is an embedding neural network that has been jointly trained with a prediction neural network, wherein the prediction neural network is configured to: receive an embedding of an input material that is generated by the embedding neural network; and process the embedding of the input material in accordance with values of a set of prediction neural network parameters to generate a prediction characterizing the input material.

In some implementations, the prediction characterizing the input material comprises a prediction for an energy of the input material.

In some implementations, the prediction characterizing the input material comprises a respective prediction of a force acting on each atom in a unit cell of the input material.

In some implementations, the prediction characterizing the input material comprises a predicted reconstruction of a chemical composition and crystal structure of the input material.

In some implementations, jointly training the embedding neural network and the prediction neural network comprises: obtaining a set of training examples, wherein each training example corresponds to a respective training material and comprises: (i) a training input characterizing the training material, and (ii) a target output of the prediction neural network; and jointly training the embedding neural network and the prediction neural network on the set of training examples.

In some implementations, jointly training the embedding neural network and the prediction neural network on the set of training examples comprises, for each training example: processing the training input of the training example using the embedding neural network to generate an embedding of the training material represented by the training input; processing the embedding of the training material using the prediction neural network to generate a predicted output; determining gradients of an objective function, with respect to the set of embedding neural network parameters of the embedding neural network and the set of prediction neural network parameters of the prediction neural network, that measures a discrepancy between: (i) the predicted output generated by the prediction neural network, and (ii) the target output specified by the training example; and adjusting values of the set of embedding neural network parameters and the set of prediction neural network parameters using the gradients.

In some implementations, selecting one or more nucleating agents for the target material, from the set of candidate nucleating agents, based at least in part on the embedding of the target material in the latent space comprises: obtaining, for each candidate nucleating agent in the set of candidate nucleating agents, a respective embedding of the candidate nucleating agent that is generated using the embedding machine learning model; determining, for each candidate nucleating agent in the set of candidate nucleating agents, a respective distance in the latent space between: (i) the embedding of the candidate nucleating agent, and (ii) the embedding of the target material; selecting the one or more nucleating agents for the target material based at least in part on the distances.

In some implementations, selecting one or more nucleating agents for the target material based at least in part on the distances comprises: ranking the candidate nucleating agents in the set of candidate nucleating agents based on their respective distances from the target material in the latent space; filtering the set of candidate nucleating agents based on the ranking to remove one or more candidate nucleating agents from the set of candidate nucleating agents; and selecting one or more of the candidate nucleating agents remaining in the set of candidate nucleating agents as nucleating agents for the target material.

In some implementations, selecting one or more candidate nucleating agents remaining in the set of candidate nucleating agents as nucleating agents for the target material comprises: determining, for each candidate nucleating agent remaining in the set of candidate nucleating agents, whether the candidate nucleating agent can stably coexist with the target material; and filtering the set of candidate nucleating agents to remove any candidate nucleating agents that cannot stably coexist with the target material from the set of candidate nucleating agents; selecting one or more of the candidate nucleating agents remaining in the set of candidate nucleating agents as nucleating agents for the target material after filtering the set of candidate nucleating agents based on: (i) the ranking of the candidate nucleating agents based on their respective distances from the target material in the latent space, and (ii) whether each candidate nucleating agent can stably coexist with the target material.

In some implementations, the embedding machine learning model comprises a neural network.

In some implementations, the embedding machine learning model comprises a graph neural network that includes a plurality of message passing neural network layers.

In some implementations, the method further comprises generating graph data representing a graph that is included in a network input to the graph neural network, wherein: the graph comprises a plurality of nodes and a plurality of edges; each node in the graph represents a respective atom in a unit cell of the target material; and each edge in the graph connects a respective pair of nodes representing a pair of atoms that are separated by less than a threshold three-dimensional spatial distance in the unit cell of the target material.

In some implementations, each node in the graph is associated with a respective set of node features that characterize the atom represented by the node; and for each node in the graph, the set of node features for the node includes a feature identifying an elemental type of the atom.

In some implementations, the method further comprises providing each of the selected nucleating agents for use in synthesizing the target material.

In some implementations, the method further comprises, for each of one or more of the selected nucleating agents, physically synthesizing the target material by a physical synthesis technique involving nucleation of the target material using the selected nucleating agent.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

The discovery and synthesis of new materials drives technological advancement in areas such as energy storage and conversion (e.g., where batteries, supercapacitors, and fuel cells rely on new materials for higher energy density and faster charging times), electronics and photonics (e.g., where new materials enable thinner, more flexible and more energy efficient devices), semiconductors (where next generation transistors, memory devices, and integrated circuits may rely on new materials), and so forth.

Various computational methods can be used to generate libraries of candidate new materials, where each candidate new material has a particular crystal structure and chemical composition. However, physical synthesis of a particular structure and composition of a compound is difficult to deliberately induce, and typically physical experiments to synthesize new materials progress through an expensive and time consuming process of trial and error.

The system described in this specification can address these issues. In particular, the system can identify a nucleating agent to be used for inducing, controlling and/or accelerating the formation of a target material with a particular chemical composition and crystal structure. The system can thus significantly accelerate the process of successfully synthesizing new materials with particular chemical compositions and crystal structures.

In particular, given a target material with a particular chemical composition and crystal structure, the system can automatically screen a library of candidate nucleating agents to identify one or more nucleating agents appropriate for use in synthesizing the target material. A candidate nucleating agent is more likely to be effective in enabling synthesis of a target material if the candidate nucleating agent has a similar crystal structure and composition to the target material, e.g., because similarity in composition reduces the likelihood of undesirable reactions or phase separations that could inhibit crystal growth or lead to defects, and because similarity in crystal structure can reduce the surface energy barrier that must be overcome for nucleation to begin. Therefore, the system can screen the library of candidate nucleating agents to identify candidate nucleating agents with a similar composition and structure to the target material.

However, numerically evaluating a similarity between the compositions and structures of materials is challenging, e.g., because different materials have different numbers of chemical constituents, different sizes of unit cell, different numbers of atoms in the unit cell, and so forth. Thus, for instance, data representing the chemical structure and composition of a first material may have a different numerical dimensionality than data representing the chemical structure and composition of a second material, and few metrics exist to measure similarity between different numerical data defined in different dimensions.

To address this issue, the system can, in some implementations, evaluate the similarity between a target material and a candidate nucleating agent by generating respective embeddings of the target material and the candidate nucleating agent in a shared latent space. The respective embeddings of the target material and the candidate nucleating agent in the shared latent space have the same dimensionality, and the system can measure a similarity between the embeddings using a conventional numerical similarity measure, e.g., based on a L1 norm or an L2 norm.

The system can generate an embedding of a material (e.g., a target material or a candidate nucleating agent) by processing data defining the chemical composition and structure of the material using an embedding machine learning model. The system can train the embedding machine learning model to generate useful embeddings that encode rich information content characterizing a material by training the embedding machine learning model using a machine learning training technique. For instance, for an embedding machine learning model implemented as an embedding neural network, the system can train the embedding neural network jointly with a prediction neural network. In particular, the system can train the embedding neural network to process chemical composition and structure data for a material to generate an embedding that, when processed by the prediction neural network, enables the prediction neural network to effectively perform a machine learning task. The machine learning task can be, e.g., to predict properties of the material, or to predict an energy of the material, or to predict forces on the atoms (or molecules) in the material, or to reconstruct the chemical structure and composition of the material.

The system can efficiently and rapidly screen a large library of candidate nucleating agents while consuming fewer computational resources, e.g., memory and computing power, than would be required for alternative approaches. For instance, an alternative approach to assessing the feasibility of using a candidate nucleating agent for synthesizing a target material may involve performing molecular dynamics simulations of a chemical system that includes the candidate nucleating agent and the target material. However, such a molecular dynamics simulation would be highly computationally intensive, e.g., as it would require simulating complex interactions between a large number of atoms over a long time scale and using short time steps. In contrast, the system described in this specification can evaluate the feasibility of a candidate nucleating agent by using an embedding machine learning model to generate embeddings of the candidate nucleating agent and the target material, and then evaluating the similarity between the respective embeddings. The number of operations required to perform two forward passes through the embedding machine learning model and then evaluate a similarity metric can be multiple orders of magnitude less than would be required to perform a molecular dynamics simulation, as described above.

In one aspect of the present disclosure there is provided a method performed by one or more computers, the method comprising: receiving data identifying a target material; generating an embedding of the target material in a latent space using an embedding machine learning model; and selecting one or more nucleating agents for the target material, from a set of candidate nucleating agents, based at least in part on the embedding of the target material in the latent space. The method can further comprise physically synthesizing the target material by a physical synthesis technique involving nucleation of the target material using the selected nucleating agent. The amount of nucleating material used may in some instances be a trace amount compared to the amount of target material being synthesized.

The target material and/or the nucleating agent(s) can each be, for example, a metal (e.g., a steel, solder or an alloy), or a ceramic (e.g., a glass-ceramic), or a polymer, or a composite, or a molecular crystal (e.g., a molecular solid comprising discrete molecules held together by intermolecular forces). In some cases, the target material and/or the nucleating agent are both crystalline solids.

In some cases, the nucleating agent(s) can be selected to cause one of a plurality of different polymorphs of the target material to be formed when the nucleating agent(s) are used in the physical synthesis of the target material. For example, the polymorph formed using the selected nucleating agent(s) can be less thermodynamically stable than a polymorph than one or more polymorphs that would be formed without the selected nucleating agent(s). In some instances, the target material can be a molecular crystal of an active pharmaceutical ingredient and the polymorph formed using the selected nucleating agent(s) can have physiochemical properties (e.g. solubility) that make it more suitable as a drug product than one or more polymorphs of the target material that would be formed without the selected nucleating agent(s).

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example nucleating agent selection system.

FIG. 2 is a flow diagram of an example process for selecting nucleating agents for use in synthesizing a target material.

FIG. 3 is a flow diagram of an example process for generating an embedding of a material, e.g., a target material or a candidate nucleating agent, using an embedding machine learning model that is implemented as a graph neural network.

FIG. 4 is a flow diagram of an example process for training an embedding machine learning model implemented as an embedding neural network.

FIG. 5 is a flow diagram of an example process for determining whether a candidate nucleating agent can stably coexist with the target material (and/or with one or more reactants to be used during synthesis of the target material).

FIG. 6 illustrates an example of determining respective distances in the latent space between: (i) the embedding of the target material, and (ii) the respective embedding of each candidate nucleating agent in the set of nucleating agents.

FIG. 7 illustrates nucleating agent Li₃PO₄ selected by the nucleating agent selection system for target material Li₂Si₂O₅.

Like reference numbers and designations in the various drawings indicate like elements.

### DETAILED DESCRIPTION

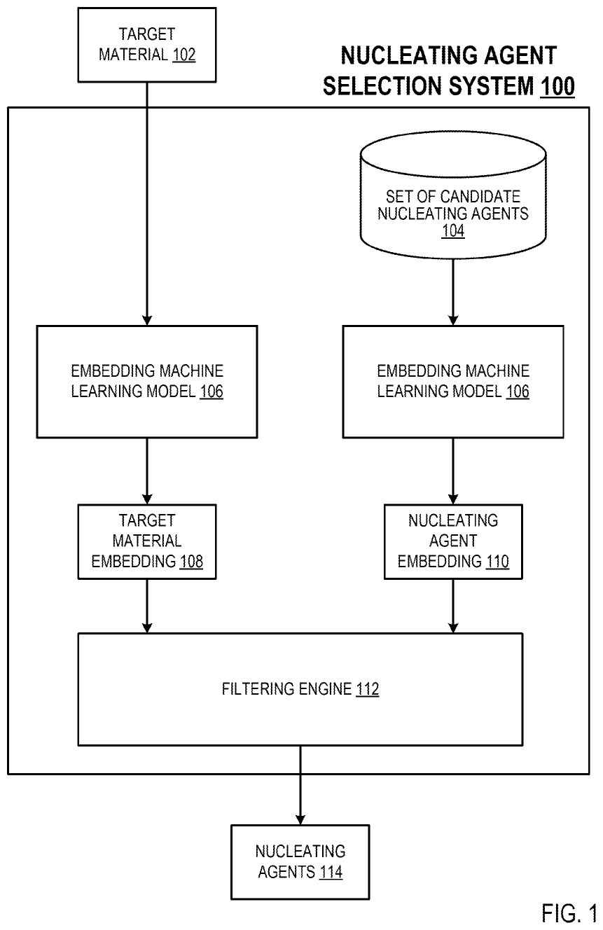

FIG. 1 shows an example nucleating agent selection system 100. The nucleating agent selection system 100 is an example of a system implemented as computer programs on one or more computers in one or more locations in which the systems, components, and techniques described below are implemented.

The system 100 is configured to process data identifying a chemical composition and a crystal structure of a target material 102 to generate data identifying one or more nucleating agents 104 that are predicted to be effective for use in synthesizing the target material 102.

The data identifying the chemical composition of the target material 102 can identify the elements included in the target material 102 and their respective proportion in the target material, e.g., by a chemical formula such as A₂B₂C₇, where A, B, and C are different elements and the numerical subscripts indicate the relative proportions of those elements in the target material.

The data identifying the crystal structure of the target material can include data that identifies, for each atom in a unit cell of the target material, a respective three-dimensional (3D) spatial position of the atom and the elemental type of the atom. The data identifying the crystal structure of the target material can further include, e.g., one or more of: data identifying lattice parameters of the unit cell (e.g., the lengths of the unit cell edges and/or the angles between the unit cell edges), lattice type (e.g., cubic, tetragonal, orthorhombic, hexagonal, trigonal, monoclinic, or triclinic), or space group (e.g., information that defines how unit cells are arranged and repeated in three-dimensional space to form the target material).

The system 100 can receive data identifying the target material 102, e.g., by way of a user interface (e.g., a graphical user interface) or an application programming interface (API) made available by the system. The data identifying the target material 102 can be provided to the system 100, e.g., by a user, or by an upstream system.

The system 100 can generate data that identifies each nucleating agent 104, e.g., by specifying a chemical composition and crystal structure of the nucleating agent. The system 100 can provide the data identifying the nucleating agents 104, e.g., by presenting data identifying the nucleating agents 104 on a display, e.g., of a user device, or by storing data identifying the nucleating agents 104 in a memory, or by transmitting data identifying the nucleating agents 104 over a data communications network.

The system includes: (i) data identifying a set of candidate nucleating agents 104, (ii) an embedding machine learning model 106, and (iii) a filtering engine 112, which are each described next (and throughout this specification).

The set of candidate nucleating agents 104 can include any appropriate number of candidate nucleating agents, e.g., at least 100, or at least 1000, or at least 100,000, or at least 1,000,000 candidate nucleating agents. In some cases, the system 100 receives the set of candidate nucleating agents 104 as an additional input, e.g., in addition to the data identifying the target material 102.

The embedding machine learning model 106 is configured process a model input that includes data identifying a material (e.g., the chemical composition of the material and the crystalline structure of the material), in accordance with values of a set of machine learning model parameters of the machine learning model, to generate an embedding of the material in a latent space. The embedding of a material can be, e.g., a vector, matrix, or other tensor having any appropriate dimensionality.

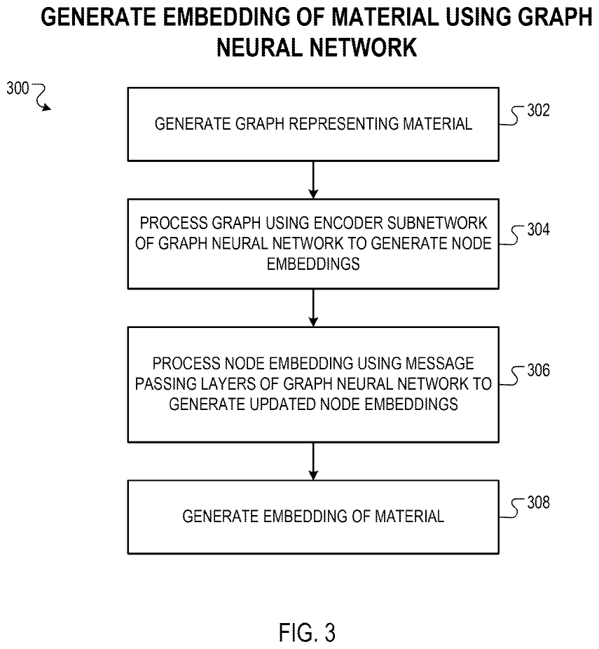

The embedding machine learning model 106 can have any appropriate machine learning model architecture. For instance, the embedding machine learning model 106 can be implemented as a neural network, e.g., a graph neural network or a convolutional neural network. An example process for generating an embedding of a material using an embedding machine learning model that is implemented as a graph neural network is described with reference to FIG. 3.

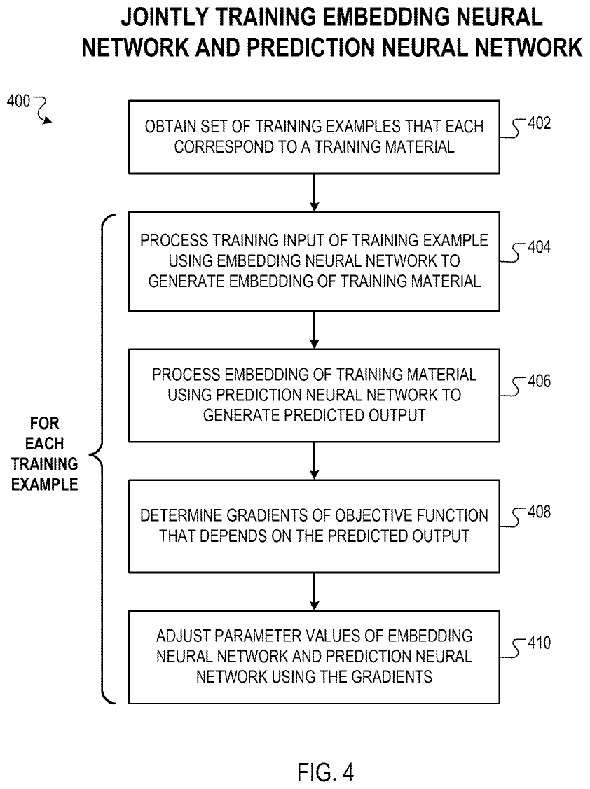

The system 100 can train the set of machine learning model parameters of the embedding machine learning model 106, by a machine learning training technique, to cause the embedding machine learning model 106 to generate embeddings that encode rich information content characterizing materials. For instance, for an embedding machine learning model 106 implemented as an embedding neural network, the system can train the embedding neural network jointly with a prediction neural network. In particular, the system 100 can train the embedding neural network to process data characterizing a material to generate an embedding that, when processed by the prediction neural network, enables the prediction neural network to effectively perform a machine learning task. An example process for jointly training an embedding neural network and a prediction neural network is described with reference to FIG. 4.

The system 100 uses the embedding machine learning model 106 to generate: (i) an embedding 108 of the target material 102, and (ii) a respective embedding 110 of each nucleating agent in the set of candidate nucleating agents 104. In particular, the system 100 processes data characterizing the target material 102 using the embedding machine learning model 106 to generate a target material embedding 108, and for each candidate nucleating agent 104, the system 100 processes data characterizing the candidate nucleating agent 104 using the embedding machine learning model to generate a corresponding nucleating agent embedding 110.

In some cases, the system 100 can precompute and store a respective nucleating agent embedding 110 for each nucleating agent in the set of nucleating agents 104, i.e., rather than recomputing the same nucleating agent embeddings 110 each time the system 100 identifies nucleating agents for a new target material 102. Precomputing the nucleating agent embeddings 110 can reduce consumption of computational resources, e.g., by preventing redundant and repeated generation of the nucleating agent embeddings 110.

The filtering engine 112 processes the target material embedding 108 and the nucleating agent embeddings 110 to select one or more nucleating agents 104 for the target material 102. In particular, the filtering engine 112 applies one or more filtering operations to the set of candidate nucleating agents, at least one of which is based on the target material embedding 108 and the nucleating agent embeddings 110, to filter the set of candidate nucleating agents 104 to remove candidate nucleating agents that satisfy filtering criteria defined by the filtering operations from the set of candidate nucleating agents 104. The filtering engine 112 can then supply any candidate nucleating agents 104 that remain in the set of candidate nucleating agents 104 after the filtering operations as nucleating agents 104 for the target material 102.

The filtering engine 112 applies at least one filtering operation that is based on the target material embedding 108 and the nucleating agent embeddings 110 to the set of candidate nucleating agents 104. In particular, the filtering engine 112 can filter the set of candidate nucleating agents 104 to maintain only those that are relatively close to the target material in the latent space, and thus have a similar chemical composition and structure as the target material. Candidate nucleating agents having a similar chemical composition and structure as the target material are more likely to be effective nucleating agents for the target material, e.g., because similarity in composition reduces the likelihood of undesirable reactions or phase separations that could inhibit crystal growth or lead to defects, and because similarity in crystal structure can reduce the surface energy barrier that must be overcome for nucleation to begin.

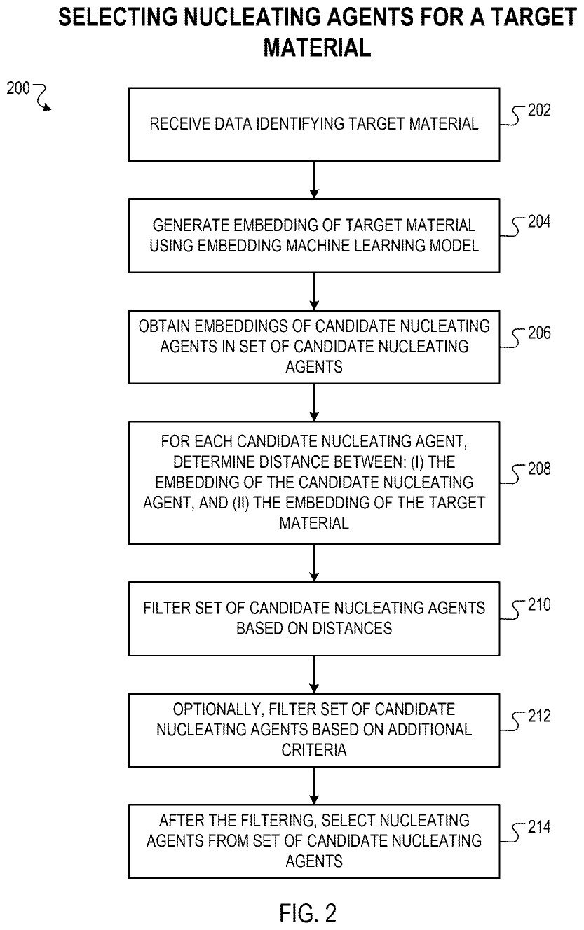

Examples of filtering operations that can be performed by the filtering engine 112 to filter the set of candidate nucleating agents 104 are described in detail below with reference to FIG. 2.

Nucleating agents 104 that are identified by the system 100 can be used in any of a variety of downstream applications. A few examples of downstream applications that involve nucleating agents 104 identified by the system 100 are described next.

In some implementations, the system 100 can perform additional computational validation of each nucleating agent 104. For instance, for each nucleating agent 104, the system 100 can perform a molecular dynamics simulation to simulate a chemical system that includes the target material 102 (or reactants used to generate the target material 102) and the nucleating agent 104 to evaluate whether the nucleating agent 104 effectively facilitates nucleation of the target material. Generally, the additional computational validation of a nucleating agent 104 consumes significantly more computational resources than are required for the system 100 to initially identify the nucleating agent 104 from the set of candidate nucleating agents 104 using the embedding machine learning model 106. The system 100 can thereby significantly reduce consumption of computational resources by initially screening the set of candidate nucleating agents 104 using the embedding machine learning model 106 and the filtering engine 112, and then performing additional computational validation only for the remaining nucleating agents.

In some implementations, for each of one or more of the nucleating agents 104, the target material 102 is physically synthesized by a physical synthesis technique involving nucleation of the target material 102 using the nucleating agent 104. The physical synthesis technique can be, e.g., a solid state reaction synthesis technique, a ceramic synthesis technique, a carbothermal synthesis technique, a combustion synthesis technique, a hydrothermal synthesis technique, a sol-gel synthesis technique, a co-precipitation synthesis technique, a precursor synthesis technique, a vapor deposition synthesis technique, or a high-pressure synthesis technique, or an electrochemical synthesis technique.

FIG. 2 is a flow diagram of an example process 200 for selecting nucleating agents for use in synthesizing a target material. For convenience, the process 200 will be described as being performed by a system of one or more computers located in one or more locations. For example, a nucleating agent selection system, e.g., the nucleating agent selection system 100 of FIG. 1, appropriately programmed in accordance with this specification, can perform the process 200.

The system receives data identifying the target material (202). The system can receive the data, e.g., from a user or from an upstream system, e.g., by way of a user interface or an API. The data identifying the target material can include data defining a chemical composition and a crystal structure of the target material.

The system processes a model input including the data identifying the target material using an embedding machine learning model and in accordance with values of a set of embedding machine learning model parameters to generate an embedding of the target material in a latent space (204). An example process for generating an embedding of a material (e.g., the target material) using an embedding machine learning model implemented as a graph neural network is described with reference to FIG. 3. An example process for training an embedding machine learning model implemented as a neural network is described with reference to FIG. 4.

The system obtains, for each candidate nucleating agent in a set of candidate nucleating agents, a respective embedding of the candidate nucleating agent in the latent space that is generated using the embedding machine learning model (206). In particular, for each candidate nucleating agent, the embedding of the candidate nucleating agent is generated by processing a model input including data identifying the candidate nucleating agent using the embedding machine learning model and in accordance with values of the set of embedding machine learning model parameters.

In some cases, the system has precomputed and stored the embeddings of the candidate nucleating agents. In these cases, the system accesses the precomputed embeddings from a memory rather than regenerating the embeddings of the candidate nucleating agents using the embedding machine learning model.

The system determines, for each candidate nucleating agent in the set of candidate nucleating agents, a respective distance in the latent space between: (i) the embedding of the candidate nucleating agent, and (ii) the embedding of the target material (208). The system can measure the distance between two embeddings in the latent space using any appropriate numerical distance measure, e.g., a Euclidean distance measure, or a distance measure based on the L1 norm, or a distance measure based on the L2 norm, and so forth.

The system filters the set of candidate nucleating agents based on the distances between the candidate nucleating agents and the target material in the latent space (i.e., as computed at step 208) (210).

For instance, the system can determine that any candidate nucleating agent that is at least a threshold distance from the target material in the latent space should be filtered (removed) from the set of candidate nucleating agents.

As another example, the system can rank the candidate nucleating agents in the set of candidate nucleating agents based on their respective distances from the target material in the latent space, e.g., from lowest distance to highest distance. The system can then filter the set of candidate nucleating agents based on the ranking to remove one or more candidate nucleating agents from the set of candidate nucleating agents. For instance, the system can determine that the N candidate nucleating agents having the smallest distances from the target material should be maintained, and all the other candidate nucleating agents should be removed from the set of candidate nucleating agents (where N is a positive integer value). As another example, the system can determine that the X % of candidate nucleating agents having the smallest distances from the target material should be maintained, and all the other candidate nucleating agents should be removed from the set of candidate nucleating agents (where X is a percentage between 0% and 100%).

The system can thus filter the set of candidate nucleating agents to maintain only those that are relatively close to the target material in the latent space, and thus have a similar chemical composition and structure as the target material. Candidate nucleating agents having a similar chemical composition and structure as the target material are more likely to be effective nucleating agents for the target material.

Optionally, the system filters the set of candidate nucleating agents based on one or more additional filtering criteria (212).

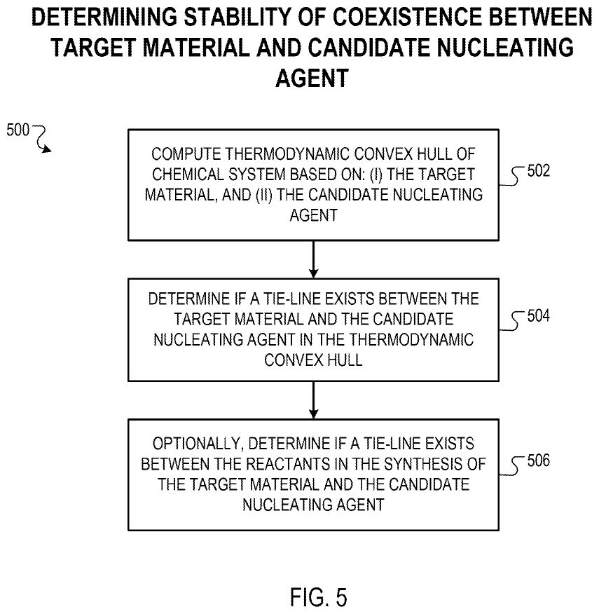

For instance, the system can determine, for each candidate nucleating agent (remaining) in the set of candidate nucleating agents, whether the candidate nucleating agent can stably coexist with the target material. The system can then filter the set of candidate nucleating agents to remove any candidate nucleating agents that cannot stably coexist with the target material. A candidate nucleating agent that cannot stably coexist with the target material may be less likely to be effective for use in synthesizing the target material, e.g., because adding the candidate nucleating agent to the target material may trigger unwanted chemical reactions. The system can determine whether a candidate nucleating agent can stably coexist with the target material (or a reactant to be used during synthesis of the target material) using any appropriate computational technique. An example process for determining whether a candidate nucleating agent can stably coexist with the target material (or a reactant to be used during synthesis of the target material) is described with reference to FIG. 5.

As another example, the system can determine, for each candidate nucleating agent (remaining) in the set of candidate nucleating agents, whether the target material can stably coexist with each reactant in a set of reactants to be used during the synthesis of the target material. A candidate nucleating agent that cannot stably coexist with a planned reactant for the target material may be less likely to be effective for use in synthesizing the target material, e.g., because the candidate nucleating agent may disrupt the synthesis reaction by reacting with the reactant. The system can determine whether a candidate nucleating agent can stably coexist with the target material (or a reactant to be used during synthesis of the target material) using any appropriate computational technique. An example process for determining whether a candidate nucleating agent can stably coexist with the target material (or a reactant to be used during synthesis of the target material) is described with reference to FIG. 5.

After filtering the set of candidate nucleating agents, the system can select some or all of the candidate nucleating agents that remain in the set of candidate nucleating agents as nucleating agents for the target material (214). The system can then provide the selected nucleating agents (i.e., that are selected by the novel operations and configuration of the system, as described in this specification), e.g., for additional computational validation using molecular dynamics simulations, or for use in physically synthesizing the target material.

FIG. 3 is a flow diagram of an example process 300 for generating an embedding of a material, e.g., a target material or a candidate nucleating agent, using an embedding machine learning model that is implemented as a graph neural network. For convenience, the process 300 will be described as being performed by a system of one or more computers located in one or more locations. For example, a nucleating agent selection system, e.g., the nucleating agent selection system 100 of FIG. 1, appropriately programmed in accordance with this specification, can perform the process 300.

The system generates graph data representing a graph that characterizes the material (302). The graph includes a set of nodes and a set of edges. Each node in the set of nodes represents a respective atom in a unit cell in the crystal structure of the material. Each edge in the set of edges connects a respective pair of nodes from the set of nodes. More specifically, each edge connects a respective pair of nodes representing a pair of atoms that are separated by less than a threshold three-dimensional (3D) spatial distance in the unit cell of the material.

The system associates each node in the set of nodes with a set of node features that characterize the atom represented by the node. More specifically, the set of node features of a node can include atom-specific features that characterize, e.g., the elemental type of the atom and the 3D spatial position of the atom, and global features that include the lattice parameters of the unit cell, the lattice type of the unit cell, and so forth.

For each node in the graph, the system processes the set of node features of the node using an encoder subnetwork of the graph neural network to generate a node embedding of the node (304). The encoder subnetwork can have any appropriate neural network architecture, e.g., the encoder subnetwork can include a sequence of fully connected neural network layers.

The system processes the node embeddings for the nodes in the graph using a sequence of message passing neural network layers of the graph neural network (306). Each message passing neural network layer is configured to process a set of input node embeddings that includes a respective node embedding for each node in the graph by a set of message passing neural network layer operations that are conditioned on the topology of the graph and are parameterized by a set of layer parameters to generate a set of output node embeddings. The “topology” of the graph refers to the way in which the nodes in the graph are connected by the edges in the graph. A message passing neural network layer can perform operations that conditioned on the topology of the graph, e.g., by, for each node in the graph, updating the node embedding of the node based only on node embeddings of neighboring nodes, i.e., that are connected to the node by an edge in the graph.

The first message passing neural network layer can receive the set of node embeddings generated by the encoder subnetwork, and each subsequent message passing neural network layer can receive the set of node embeddings generated by the preceding message passing neural network layer.

The system generates an embedding of the material based on the node embeddings generated by the final message passing neural network layer in the sequence of message passing neural network layers (308). For instance, the system can generate the embedding of the material by aggregating (e.g., summing or averaging) the node embeddings generated by the final message passing neural network layer.

FIG. 4 is a flow diagram of an example process 400 for training an embedding machine learning model implemented as an embedding neural network. For convenience, the process 400 will be described as being performed by a system of one or more computers located in one or more locations. For example, a nucleating agent selection system, e.g., the nucleating agent selection system 100 of FIG. 1, appropriately programmed in accordance with this specification, can perform the process 400.

The system obtains a set of training examples that each correspond to a respective training material and each include: (i) a training input that includes data defining the chemical composition and crystal structure of the training material, and (ii) a target output (402). Chemical compositions and crystal structures for training materials can be obtained from a number of publically accessible databases, such as the Cambridge Structural Database (CSD) or Inorganic Crystal Structure Database (ICSD).

In some implementations, each training example includes a target output that defines an energy (e.g., a total energy) of the training material. The system can determine the energy of the material, e.g., using a density functional theory (DFT) calculation.

In some implementations, each training example includes a target output that defines a respective force acting on each atom in a unit cell of the training material (e.g., the force on each atom in a single unit cell of the training material, without taking into account the interactions with atoms in other unit cells). The system can determine the forces acting on the atoms in the unit cell of the training material, e.g., using a DFT calculation. A force acting on an atom can be represented, e.g., as a 3D vector defining a respective force in the x-, y-, and z-directions.

In some implementations, each training example includes a target output that is the same as some or all of the training input of the training example. For example, the target output can comprise the crystal structure of the training material included in the training input.

The system jointly trains the embedding neural network and a prediction neural network on the set of training examples. Jointly training the embedding neural network and the prediction neural network on a training example is described next with reference to steps 404-410.

The system processes the training input of the training example using the embedding neural network to generate an embedding of the training material represented by the training input (404).

The system processes the embedding of the training material using the prediction neural network to generate a predicted output (406). The predicted output defines a prediction for the target output specified by the training example and can include one or more of: a predicted energy of the training material, predicted forces acting on the atoms in the unit cell of the training material, or a predicted reconstruction of the training input processed by the embedding neural network.

The prediction neural network can have any appropriate neural network architecture that enables the prediction neural network to perform its described functions. In particular, the prediction neural network can include any appropriate types of neural network layers (e.g., fully connected layers, attention layers, convolutional layers, and so forth) in any appropriate number (e.g., 3 layers, 5 layers, or 10 layers), and connected in any appropriate configuration (e.g., as a directed graph of layers). For instance, in a particular example, the prediction neural network can include a sequence of fully connected neural network layers.

The system determines gradients of an objective function, with respect to a set of embedding neural network parameters of the embedding neural network and a set of prediction neural network parameters of the prediction neural network, that measures a discrepancy between: (i) the predicted output generated by the prediction neural network, and (ii) the target output specified by the training example (408). The objective function can measure the discrepancy between the predicted output and the target output, e.g., using an L1 norm or an L2 norm or in any other appropriate manner. The system can determine the gradients of the objective function, e.g., using backpropagation.

The system adjusts values of the set of embedding neural network parameters and the set of prediction neural network parameters using the gradients (410). For instance, the system can adjust the values of the set of embedding neural network parameters and the set of prediction neural network parameters using the update rule of any appropriate gradient descent optimization algorithm, e.g., RMSprop or Adam.

FIG. 5 is a flow diagram of an example process 500 for determining whether a candidate nucleating agent can stably coexist with the target material (or a reactant to be used during synthesis of the target material). For convenience, the process 500 will be described as being performed by a system of one or more computers located in one or more locations. For example, a nucleating agent selection system, e.g., the nucleating agent selection system 100 of FIG. 1, appropriately programmed in accordance with this specification, can perform the process 500.

The system determines a thermodynamic convex hull (or phase diagram) of a chemical system that includes the target material and the candidate nucleating agent (502). For example, if the target material has chemical formula A₂B₂C₇ and the candidate nucleating agent has the formula X₂C₃, the system can determine the thermodynamic convex hull of the chemical system A—B—X—C. The system can determine the thermodynamic formation energy of the constituents of the chemical system, e.g., using DFT or a machine learning model. Optionally, the chemical system can further include each reactant in a set of reactants to be used during synthesis of the target material.

The system determines that the candidate nucleating agent can stably coexist with the target material if the thermodynamic convex hull includes a tie-line between the candidate nucleating agent and the target material (504). Other methods of determining whether the target material can stably coexist with the target material can of course be used.

Optionally, the system can determine that the candidate nucleating agent can stably coexist with a reactant to be used during the synthesis of the target material if the thermodynamic convex hull includes a tie-line between the target material and the reactant (506).

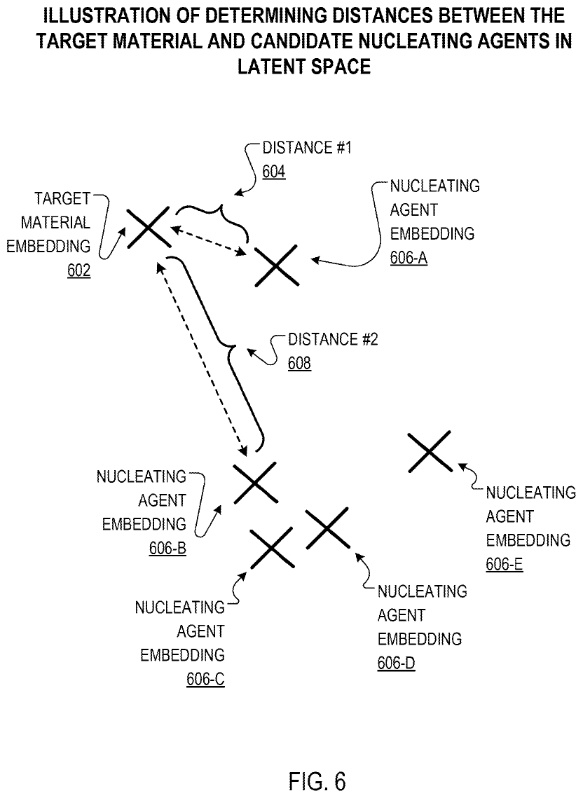

FIG. 6 illustrates an example of determining respective distances in the latent space between: (i) the embedding of the target material 602, and (ii) the respective embedding of each candidate nucleating agent 606-A-E in the set of candidate nucleating agents. The distance 604 between the target material 602 and the nucleating agent embedding 606-A is less than the distance 608 between the target material 602 and the nucleating agent 606-B. Therefore the nucleating agent 606-A is likely to have a higher compositional and structural similarity to the target material than the nucleating agent 606-B, and the nucleating agent selection system is more likely to select nucleating agent 606-A than nucleating agent 606-B.

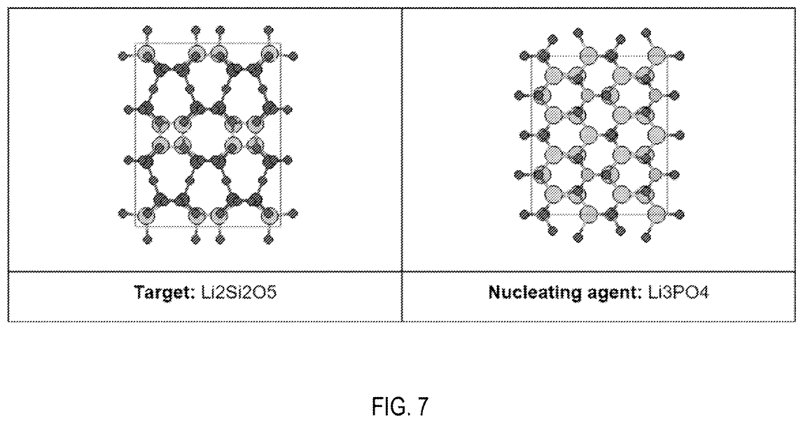

FIG. 7 illustrates nucleating agent Li₃PO₄ selected by the nucleating agent selection system for target material Li₂Si₂O₅.

Höland et al. Phosphorus Research Bulletin 19 (2005) 36 reports Li3PO4 as a nucleating agent for successfully inducing the Li2Si2O5 phase in SiO2-Li2O-Al2O3-K2O-ZrO2 based functional glass-ceramics. The nucleating agent selection system described in this specification identified Li3PO4 among the top four nucleating agents recommended, alongside Li4P2O7, Li2SiO3 and Na2Si2O5.

DeCeanne et al. Journal of Non-Crystalline Solids, 591 (2022) 121714 reports various nucleating agents in glass ceramic systems, e.g., Nb2O5 addition to Na2O-Al2O3-SiO2 glass system induces nucleation of NaNbO3. The nucleating agent selection system described in this specification ranks Nb2O5 at the top among all binary oxides for selectively nucleating NaNbO3 phase.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
receiving data identifying a target material;
generating an embedding of the target material in a latent space using an embedding machine learning model; and
selecting one or more nucleating agents for the target material, from a set of candidate nucleating agents, based at least in part on the embedding of the target material in the latent space.

2. The method of claim 1, wherein the data identifying the target material comprises: (i) data identifying a chemical composition of the target material, and (ii) data identifying a crystal structure of the target material.

3. The method of claim 1, wherein generating the embedding of the target material in the latent space using the embedding machine learning model comprises:
processing a model input comprising the data identifying the target material using the embedding machine learning model and in accordance with values of a set of embedding machine learning model parameters to generate the embedding of the target material.

4. The method of claim 1, wherein the embedding machine learning model is an embedding neural network that has been jointly trained with a prediction neural network, wherein the prediction neural network is configured to:
receive an embedding of an input material that is generated by the embedding neural network;
process the embedding of the input material in accordance with values of a set of prediction neural network parameters to generate a prediction characterizing the input material.

5. The method of claim 4, wherein the prediction characterizing the input material comprises a prediction for an energy of the input material.

6. The method of claim 4, wherein the prediction characterizing the input material comprises a respective prediction of a force acting on each atom in a unit cell of the input material.

7. The method of claim 4, wherein the prediction characterizing the input material comprises a predicted reconstruction of a chemical composition and crystal structure of the input material.

8. The method of claim 4, wherein jointly training the embedding neural network and the prediction neural network comprises:
obtaining a set of training examples, wherein each training example corresponds to a respective training material and comprises: (i) a training input characterizing the training material, and (ii) a target output of the prediction neural network; and
jointly training the embedding neural network and the prediction neural network on the set of training examples.

9. The method of claim 8, wherein jointly training the embedding neural network and the prediction neural network on the set of training examples comprises, for each training example:
processing the training input of the training example using the embedding neural network to generate an embedding of the training material represented by the training input;
processing the embedding of the training material using the prediction neural network to generate a predicted output;
determining gradients of an objective function, with respect to the set of embedding neural network parameters of the embedding neural network and the set of prediction neural network parameters of the prediction neural network, that measures a discrepancy between: (i) the predicted output generated by the prediction neural network, and (ii) the target output specified by the training example; and
adjusting values of the set of embedding neural network parameters and the set of prediction neural network parameters using the gradients.

10. The method of claim 1, wherein selecting one or more nucleating agents for the target material, from the set of candidate nucleating agents, based at least in part on the embedding of the target material in the latent space comprises:
obtaining, for each candidate nucleating agent in the set of candidate nucleating agents, a respective embedding of the candidate nucleating agent that is generated using the embedding machine learning model;
determining, for each candidate nucleating agent in the set of candidate nucleating agents, a respective distance in the latent space between: (i) the embedding of the candidate nucleating agent, and (ii) the embedding of the target material;
selecting the one or more nucleating agents for the target material based at least in part on the distances.

11. The method of claim 10, wherein selecting one or more nucleating agents for the target material based at least in part on the distances comprises:
ranking the candidate nucleating agents in the set of candidate nucleating agents based on their respective distances from the target material in the latent space;
filtering the set of candidate nucleating agents based on the ranking to remove one or more candidate nucleating agents from the set of candidate nucleating agents; and
selecting one or more of the candidate nucleating agents remaining in the set of candidate nucleating agents as nucleating agents for the target material.

12. The method of claim 11, wherein selecting one or more candidate nucleating agents remaining in the set of candidate nucleating agents as nucleating agents for the target material comprises:
determining, for each candidate nucleating agent remaining in the set of candidate nucleating agents, whether the candidate nucleating agent can stably coexist with the target material; and
filtering the set of candidate nucleating agents to remove any candidate nucleating agents that cannot stably coexist with the target material from the set of candidate nucleating agents;
selecting one or more of the candidate nucleating agents remaining in the set of candidate nucleating agents as nucleating agents for the target material after filtering the set of candidate nucleating agents based on:
(i) the ranking of the candidate nucleating agents based on their respective distances from the target material in the latent space, and
(ii) whether each candidate nucleating agent can stably coexist with the target material.

13. The method of claim 1, wherein the embedding machine learning model comprises a neural network.

14. The method of claim 13, wherein the embedding machine learning model comprises a graph neural network that includes a plurality of message passing neural network layers.

15. The method of claim 13, further comprising generating graph data representing a graph that is included in a network input to the graph neural network, wherein:
the graph comprises a plurality of nodes and a plurality of edges;
each node in the graph represents a respective atom in a unit cell of the target material; and
each edge in the graph connects a respective pair of nodes representing a pair of atoms that are separated by less than a threshold three-dimensional spatial distance in the unit cell of the target material.

16. The method of claim 15, wherein each node in the graph is associated with a respective set of node features that characterize the atom represented by the node;
wherein for each node in the graph, the set of node features for the node includes a feature identifying an elemental type of the atom.

17. The method of claim 1, further comprising providing each of the selected nucleating agents for use in synthesizing the target material.

18. The method of claim 1, further comprising, for each of one or more of the selected nucleating agents, physically synthesizing the target material by a physical synthesis technique involving nucleation of the target material using the selected nucleating agent.

19. A system comprising:
one or more computers; and
one or more storage devices communicatively coupled to the one or more computers, wherein the one or more storage devices store instructions that, when executed by the one or more computers, cause the one or more computers to perform operations comprising:
receiving data identifying a target material;
generating an embedding of the target material in a latent space using an embedding machine learning model; and
selecting one or more nucleating agents for the target material, from a set of candidate nucleating agents, based at least in part on the embedding of the target material in the latent space.

20. One or more non-transitory computer storage media storing instructions that when executed by one or more computers cause the one or more computers to perform operations comprising:
receiving data identifying a target material;
generating an embedding of the target material in a latent space using an embedding machine learning model; and
selecting one or more nucleating agents for the target material, from a set of candidate nucleating agents, based at least in part on the embedding of the target material in the latent space.
